(19) United States
(12) Patent Application Publication
(10) Pub. No.: US 2014/0257429 A1
(43) Pub. Date: Sep. 11, 2014

(54) Stimulation Apparatus

(71) Applicant: Louise Mohn, (US)

(72) Inventors: Louise Mohn, Oslo (NO); Ole Brix, Bergen (NO); Inge Klepsvik, Bergen (NO); Bard Henriksen, Bergen (NO)

(21) Appl. No.: 14/357,332

(22) PCT Filed: Nov. 14, 2012
(86) PCT No.: PCT/EP2012/072621

§ 371 Date: May 9, 2014

(30) Foreign Application Priority Data
Nov. 14, 2011 (GB) 1119608.6

(51) Int. Cl. A61N 1/36

(52) U.S. Cl. CPC A61N 1/36003
USPC 607/48; 607/62

(57) ABSTRACT

An apparatus (40) comprises means (402) for applying electrical stimulation to a human or animal body via a pair of electrodes (50). The apparatus further comprises means (406, 410) for measuring impedance of the body between the pair of electrodes (50).

### FIELD OF THE INVENTION

The present invention relates to an apparatus for applying electrical stimulation to a human or animal body. The apparatus is also capable of measuring the impedance of the human or animal body.

### BACKGROUND OF THE INVENTION

For a variety of therapeutic applications, several treatment modalities are currently known in the art including electrical stimulation, heat therapy and thermostimulation.

Electrical stimulation involves the application of an electrical current to a single muscle or a group of muscles through one or more stimulation pads that are temporarily attached to the skin. The resulting muscle contraction can produce a variety of effects from strengthening injured muscles and reducing oedema to relieving pain and promoting healing. The pads are usually quite small and typically powered with a battery. This results in the application of a small amount of power and a low treatment depth of the resulting electric field. The shallow depth of the electric field generated by conventional electrical stimulation systems limits performance and patient benefit. Some systems have attempted to address this limitation by applying more current, often from a line or mains supply source. However, the small size of conventional electrical stimulation pads is such that on the application of larger amounts of power, i.e. the use of higher currents, patients often report the experience of pain or discomfort.

Heat therapy involves the application of heat to the body. Heat therapy is very useful as it has a number of effects such as relaxation of muscle spasm and increased blood flow that promotes healing. However, combination therapy, i.e. the synergistic use of other modalities such as massage, ultrasound and/or electrical stimulation has been found to be more effective than heat therapy alone.

Thermostimulation is one such combination therapy that involves the use of heat therapy and electrical stimulation simultaneously. With thermostimulation, the healing benefits of heat are provided along with the strengthening, toning, pain relieving and healing benefits of electrical stimulation. Moreover, the application of heat has been found effective in that it allows the patient to tolerate higher currents. This yields higher electric field strengths, greater depths of penetration and, therefore, more positive results than could be achieved with electrical stimulation without heat. Thermostimulation can be performed using pads that are temporarily attached to the skin.

The inventors have identified a need to provide improved apparatuses for electrical stimulation or thermostimulation.

### SUMMARY OF THE INVENTION

In accordance with the invention, there is provided an apparatus comprising: means for applying electrical stimulation to a human or animal body via a pair of electrodes; and means for measuring impedance of the body between the pair of electrodes.

Using the same pair of electrodes to apply electrical stimulation and to measure impedance of the body results in an apparatus that is compact and simple to manufacture. Furthermore, this also results in a stimulation pad that is compact and simple to manufacture. Yet further, this simplifies use of the apparatus, by avoiding the need to apply separate sets of electrodes to the body to enable electrical stimulation and impedance measurement.

The apparatus preferably comprises means for controlling the electrical stimulation based upon the impedance measured by the means for measuring impedance. Using the same pair of electrodes to apply electrical stimulation and to measure impedance of the body allows the electrical stimulation to be controlled based upon the local impedance at precisely the region of the body to which stimulation is applied. Preferably, the means for controlling the electrical stimulation is operable to adjust the amplitude of the electrical stimulation applied to the body. More preferably, the means for controlling the electrical stimulation is operable to adjust the amplitude of the electrical stimulation applied to the body to compensate for variations in the impedance measured by the means for measuring impedance. Preferably, the means for controlling the electrical stimulation is operable to stop electrical stimulation being applied to the body if the impedance measured by the means for measuring impedance is less than a first threshold impedance value or greater than a second threshold impedance value.

The apparatus preferably further comprises a pad for placement on the body, wherein the pad comprises the pair of electrodes. Providing the pair of electrodes in a single pad simplifies use of the stimulation apparatus, since only the pad needs to be placed on the body in order to apply electrical stimulation and measure impedance. Hence, the need to apply to the body a separate device specifically for the purpose of measuring impedance is avoided.

The means for measuring impedance preferably comprises a first means for measuring voltage, the first means for measuring voltage being operable to measure the voltage between the pair of electrodes. The means for measuring impedance preferably further comprises a means for measuring current, the means for measuring current being operable to measure the current through the electrodes. The means for measuring current comprises: a resistor arranged to be connected in series with the electrodes; and a second means for measuring voltage, the second means for measuring voltage being operable to measure the voltage across the resistor. The means for measuring impedance preferably further comprises means for calculating impedance using the voltages measured by the first and second means for measuring voltage. The means for measuring current is preferably operable to measure current through the electrodes whilst the means for applying electrical stimulation is applying electrical stimulation to the body. The means for measuring impedance preferably comprises means for applying a measurement signal to the body, the means for measuring impedance being operable to measure impedance of the body whilst the measurement signal is being applied. Preferably, the amplitude of the measurement signal is chosen to prevent muscle contraction.

### BRIEF DESCRIPTION OF THE DRAWINGS

Preferred features of the invention will now be described, purely by way of example, with reference to the accompanying drawings, wherein like elements are indicated using like reference signs, and in which:

FIG. 1 is a schematic diagram of a stimulation system;

FIG. 2 is an exploded view of a stimulation pad for use with the stimulation system of FIG. 1;

FIG. 3 is a top plan view of a circuit for use with the stimulation pad of FIG. 2;

FIG. 4 is a bottom plan view of the circuit shown in FIG. 3;

FIG. 5 is a schematic diagram of a connector for the circuit shown in FIG. 3;

FIG. 6 is a circuit diagram of a heating element for the circuit shown in FIG. 3;

FIG. 7 is schematic diagram of a stimulation circuit for use with the stimulation system of FIG. 1;

FIG. 8 is a circuit diagram of a signal generator;

FIG. 9 is a graph of voltage against time illustrating an example of the use of the stimulation system of FIG. 1 to apply electrical stimulation and measure impedance; and

FIG. 10 is a graph of voltage against time illustrating another example of the use of the stimulation system of FIG. 1 to apply electrical stimulation and measure impedance.

### DETAILED DESCRIPTION

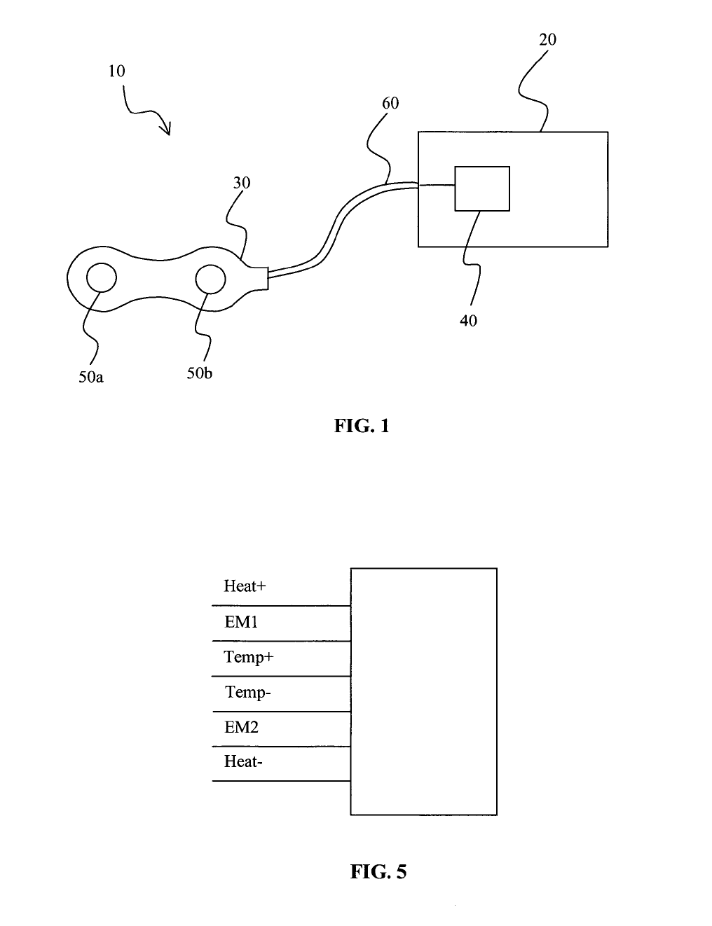

FIG. 1 shows a stimulation system 10. The stimulation system 10 comprises a console 20 and a stimulation pad 30. The console 20 comprises a stimulation circuit 40. The stimulation pad 30 is electrically connected (and, preferably, detachably connected) to the stimulation circuit 40 by a cable 60. The stimulation pad comprises one or more electrodes 50a, 50b. In use, the stimulation pad is placed upon a human or animal body. The stimulation circuit 40 is operable to apply electrical stimulation to the body via the electrodes 50 in the stimulation pad 30. The stimulation circuit 40 is also operable to measure the impedance of the body between the same electrodes.

In addition to applying electrical stimulation to the body via the stimulation circuit 40, the stimulation system 10 may also be able to apply heat to the body. That is, the stimulation system 10 can be a thermostimulation system. Such a thermostimulation system is preferably operable to apply heat and electrical stimulation to the body simultaneously or independently of each other.

For the sake of simplicity, the invention will be described with reference to an example of a stimulation pad 30 that comprises two electrodes 50. An example of a suitable stimulation pad is disclosed in the applicant's earlier patent application, WO 2011/064527, the entire contents of which are incorporated by reference herein. WO 2011/064527 describes a stimulation pad having two elongate substantially parallel electrodes for electrical stimulation, each preferably moulded from carbon loaded silicone. The electrodes are then over-moulded, to hold the electrodes in position relative to one another, thereby providing a single moulded assembly. A heating element is positioned on the moulded assembly and held in place with a layer of silicone.

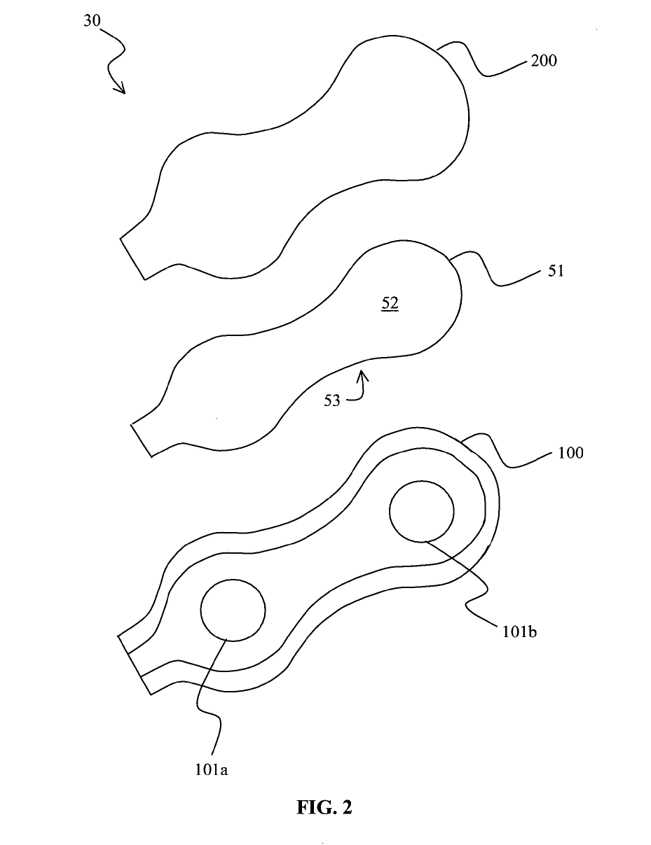

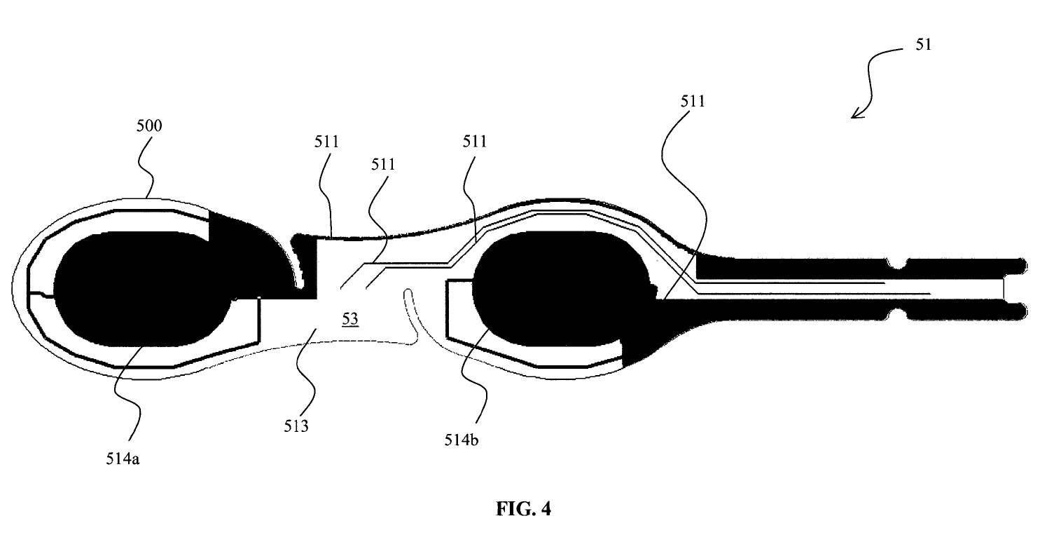

Another example of a suitable pad is the novel pad described below, with reference to FIGS. 2 to 6. In this example, the stimulation pad 30 comprises a circuit enclosed within a protective casing. FIG. 2 shows an exploded view of the stimulation pad 30. The circuit 51 fits into a casing body 100. The casing body 100 may be moulded from a plastics material. The casing body 100 comprises areas 101a, 101b of conducting material 101. The areas of conducting material 101 may comprise a polymer mixed with graphite. When fitted, a first surface 53 of the circuit 51 faces towards the casing body 100 and is aligned so each electrode 514a, 514b (shown in FIG. 4) is in electrical contact with a respective conducting area 101a, 101b. A cover 200 is provided on top of the casing body 100, thereby enclosing the circuit 51. The casing body 100 and cover 200 protect the circuit 51 against the ingress of water, which could cause the circuit 51 to malfunction. In use, the stimulation pad 30 is placed on the body of a user. The conducting areas 101 conduct an electrical current from the electrodes 514 to the body of the user.

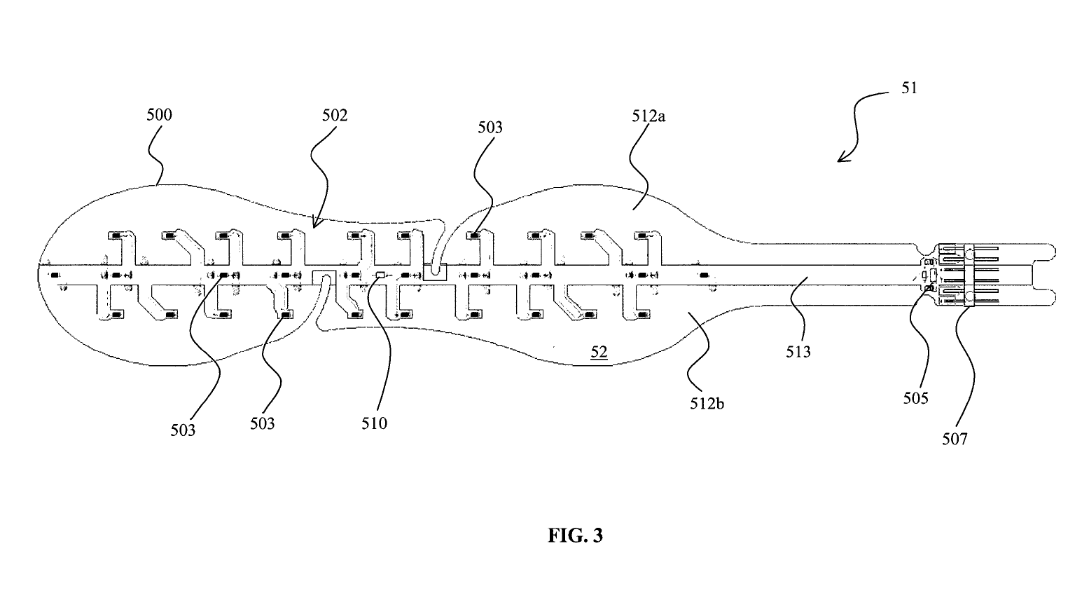

FIG. 3 is a top plan view of the circuit 51 of the stimulation pad 30 shown in FIG. 2. FIG. 4 is a bottom plan view of the circuit 51. As shown in FIG. 3 and FIG. 4, the circuit 51 comprises a substrate 500. The substrate 500 has a first surface 53 (shown in FIG. 4) and a second surface 52 (shown in FIG. 3), wherein the first surface 53 has an opposite orientation to the second surface 52. The circuit 51 further comprises a heating element 502 and one or more electrodes 514. The circuit 51 can further comprise electronic components including a temperature sensor 510, a visual indicator 505 and a connector 507.

Electrical conductors 511, 512 are patterned on each surface 52, 53 of the substrate 500 to form electrical connections between the components of the circuit 51. As used herein, the term “patterned” is preferably understood to describe the result of a process whereby an electrically conducting region having a predefined shape is formed upon a surface of the substrate 500. The conductors are illustrated by the grey shaded areas in FIGS. 3 and 4. The conductors on the first surface 53 are denoted by reference numeral 511 in FIG. 4, whilst the conductors on the second surface 52 are denoted by reference numeral 512 in FIG. 3. One or more electrodes 514 are also patterned on the first surface 53 of the substrate 500. The electrodes 514 are also illustrated by grey shaded areas in FIG. 4, since the electrodes 514 are preferably formed from the same electrically conducting material as the conductors 511. Insulating regions that do not comprise a conductor are illustrated in FIGS. 3 and 4 by the unshaded areas denoted by reference numeral 513.

The electronic components 502, 505, 507, 510, conductors 511, 512 and electrodes 514 are provided on both surfaces 52, 53 of the substrate 500. The electrodes 514 are formed on the first surface 53, whilst the heating element 502 is formed on the second surface 52. In use, the heating element 502 faces away from the skin of the user and the electrodes 514 face towards the skin. The temperature sensor 510, visual indicator 505 and connector 507 are also preferably provided on the second surface 52. Since electronic components 502, 505, 507, 510, conductors 511, 512 and electrodes 514 are provided on both surfaces of the substrate 500, the substrate 500 should have electrically insulating properties in order to prevent unwanted electrical conduction between components and conductors on different surfaces.

The circuit 51 comprises a connector 507 to allow the circuit to be electrically connected to the cable 60 (shown in FIG. 1) and thereby connected to the console 20 (also shown in FIG. 1). The connector 507 is preferably provided on the second surface 52. The connector 507 is preferably a surface-mount connector. The connector 507 comprises connection pins, which can be connected to a corresponding connector on the cable 60. In an example, the connector 507 comprises six connection pins, as illustrated in FIG. 5. The pins labelled ‘Heat+’ and ‘Heat−’ are connected to the heating element 502. The pins labelled ‘Temp+’ and ‘Temp−’ are connected to the temperature sensor 510. The pins labelled ‘EM1’ and ‘EM2’ are connected to the electrodes 514.

The heating element 502 preferably comprises a plurality of resistors 503 and one or more conductors 512. The resistors 503 are distributed across the second surface 52 of the substrate 500. For the sake of clarity, only three resistors 503 are labelled in FIG. 3; however, it can be seen that the circuit comprises many more resistors, each of which is illustrated as a small black rectangle in FIG. 3. The resistors 503 are electrically connected to each other by the conductors 512. In the example illustrated in FIG. 3, conductor 512a is connected to the ‘Heat-’ pin of the connector 507 such that, in use, the conductor 512a operates as a negative voltage supply rail. Similarly, conductor 512b is connected to the ‘Heat+’ pin of the connector 507 such that, in use, the conductor 512b operates as a positive voltage supply rail.

When a voltage is applied across the resistors 503, power is dissipated as heat. The positive and negative supply voltages are supplied to the resistors 503 by the pins labelled ‘Heat+’ and ‘Heat−’ respectively in the connector 507. The resistors 503 are soldered to the conductors 512, and are thereby electrically connected to the connector 507. The power dissipated by each resistor 503 is defined as:

P=I²R (1)

where P is the power dissipated (measured in watts), I is the current through the resistor (measured in amperes), and R is the resistance of the resistor (measured in ohms).

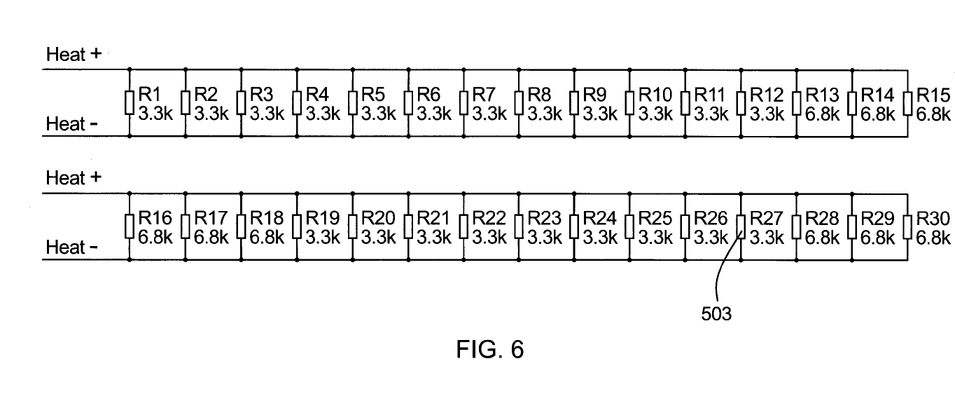

In an example, thirty resistors 503 are distributed over the area of the second surface 52. FIG. 6 is a circuit diagram of this example. The resistance values of the resistors 503 range from 3.3 kilohms to 6.8 kilohms in order to avoid localised areas generating more heat than surrounding regions. FIG. 6 shows that the resistors 503 are connected in parallel, but it will be appreciated that they could also be connected in series or in a combination of series and parallel connections. In an example, a direct current input voltage of twenty-four volts is applied across the resistors 503. The present invention is not limited to any particular input voltage or resistance values.

The temperature sensor 510 is mounted on the second surface 52 of the substrate 500, using surface-mount technology. The temperature sensor 510 is preferably mounted at the point equidistant between the electrodes 514a, 514b. This is to give an indication of the temperature near the region where electrical stimulation is applied, although the temperature sensor 510 could be placed at any other suitable point on the second surface 52. The positive and negative supply voltages for the temperature sensor 510 are supplied by the pins labelled ‘Temp+’ and ‘Temp−’ respectively in the connector 507. The temperature sensor 510 is coupled to the connector 507 by the conductors 511 patterned on the first surface 53 of the substrate 500. Vias through the substrate 500 connect the conductors 511 on the first surface 53 to the temperature sensor 510 and connector 507 that are mounted on the second surface 52. The temperature sensor 510 can be a resistance thermometer or a thermocouple. The temperature sensor is preferably a platinum resistance thermometer (PRT), and is more preferably a Pt1000 element. A Pt1000 element is preferable due to its high accuracy.

An electrical stimulation current is delivered from the console 20 to the electrodes 514a, 514b by the pins of the connector 507 labelled ‘EM1’ and ‘EM2’ respectively. The electrodes 514 are coupled to the connector 507 by the conductors 511 patterned on the first surface 53. Vias through the substrate 500 connect the conductors 511 on the first surface 53 to the connector 507 that is mounted on the second surface 52.

Other electronic components could be mounted on the substrate 500 and, preferably, mounted on the second surface 52 of the substrate. For example, logic components such as a programmable logic device, microprocessor or microcontroller could be mounted on the substrate 500. Such logic components could be used to control the heat and/or electrical stimulation that is applied to a user. As another example, one or more sensors could be mounted on the substrate 500, in addition to the temperature sensor 510. As shown in FIG. 3, a visual indicator 505 can be mounted on the second surface 52 of the substrate 500. The visual indicator 505 is preferably a light emitting diode.

As mentioned previously, in use, the heating element 502 faces away from the skin of the user and the electrodes 514 face towards the skin. Thus, heat generated in the heating element 502 on the second surface 52 is conducted through the substrate 500 to the first surface 53, and is subsequently conducted to the body of a user through the casing body 100 of the stimulation pad 30.

The example of a stimulation pad 30 that is described above with reference to FIGS. 2 to 6 is intended purely to illustrate an example of a stimulation pad that is suitable for use with the inventive stimulation circuit 40 that is described below. The stimulation circuit 40 can be used with other suitable stimulation pads.

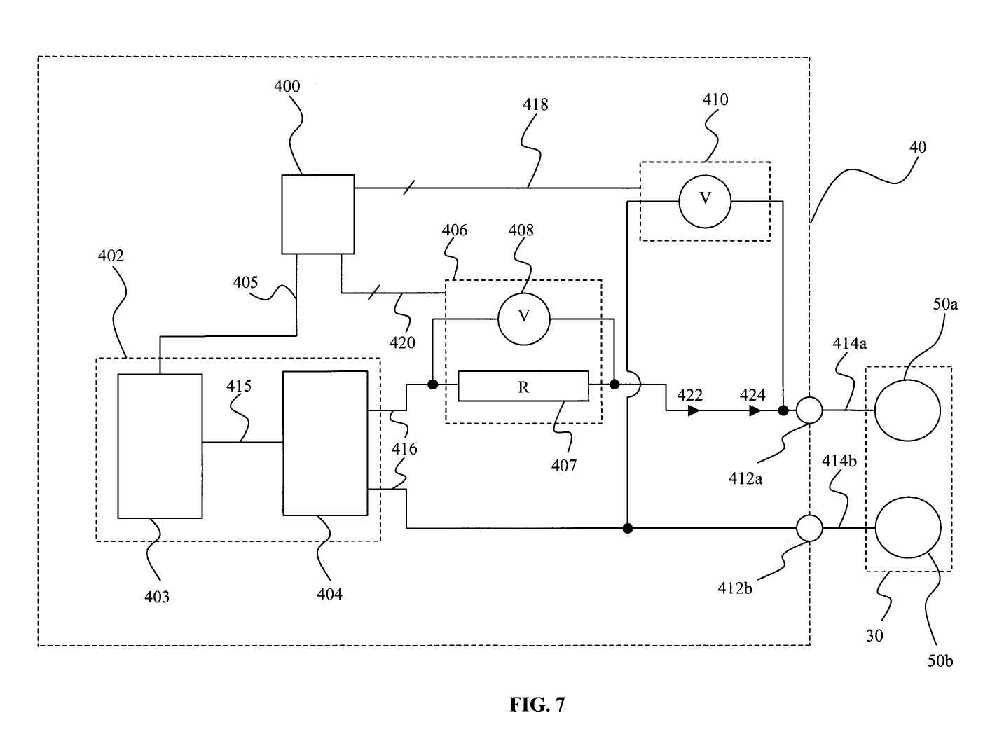

FIG. 7 is a schematic diagram of the stimulation circuit 40 connected to the electrodes 50 of a stimulation pad 30. The stimulation circuit 40 comprises a controller 400, a signal generator 402, a first means for measuring voltage 410 and a means for measuring current 406. The stimulation circuit 40 further comprises two terminals 412a, 412b for electrical connection with a respective electrode 50a, 50b of a stimulation pad 30, via connecting wires 414a, 414b. The connecting wires 414a, 414b are contained within the cable 60 (shown in FIG. 1). The terminals 412a, 412b preferably allow the cable 60 and pad 30 to be detached from the stimulation circuit 40, such that the stimulation circuit 40 and pad 30 can be supplied separately.

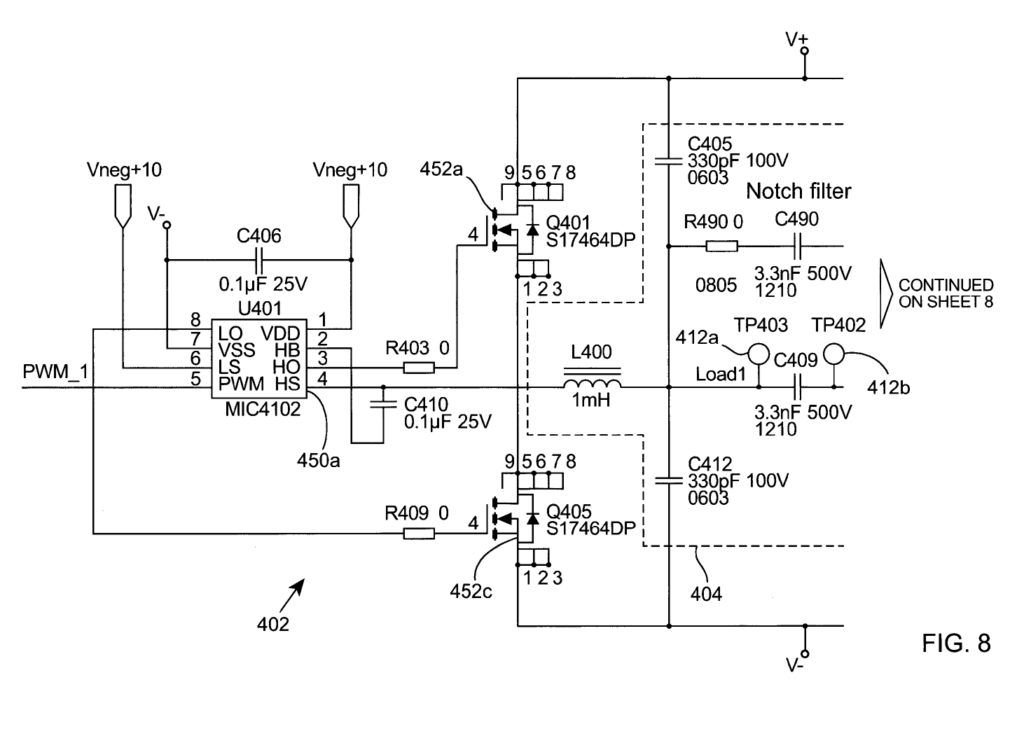

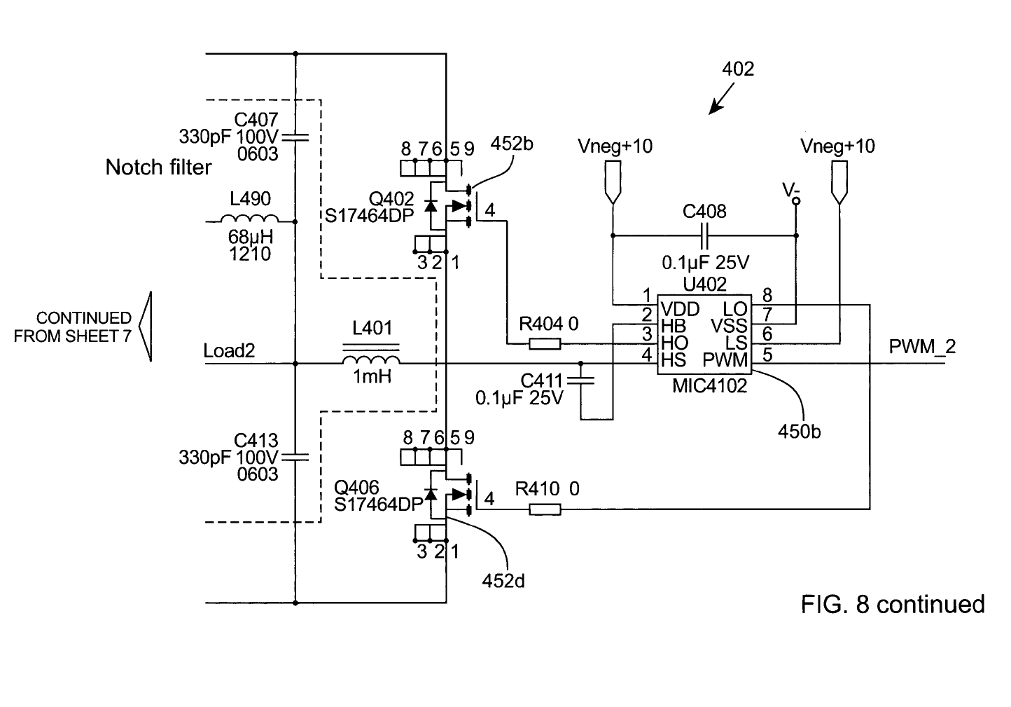

The signal generator 402 can comprise an amplifier 403 and a filter 404. FIG. 8 is a circuit diagram of a preferred embodiment of the signal generator 402. In the circuit shown in FIG. 8, the signal generator 402 comprises a class-D amplifier and a notch filter 404. A class-D amplifier is preferable because of its high power efficiency, i.e. the power that is transmitted to the body via the pad 30 is relatively high, and the heat dissipated in the amplifier is relatively low. A further advantage of using a class-D amplifier is that it can receive a pulse-width modulated digital input signal from the controller 400, which avoids the need to convert the output of the controller 400 from the digital domain to the analogue domain before amplification. The class-D amplifier preferably comprises a pair of half bridge drivers 450a, 450b and four field effect transistors 452a, 452b, 452c, 452d. As shown in FIG. 8, the class-D amplifier comprises a pair of MIC4102 half bridge MOSFET driver integrated circuits, manufactured by Micrel, Inc., and four SI7464 MOSFET integrated circuits. The filter 404 is a passive filter, which comprises one or more resistors, one or more capacitors and one or more inductors. It will be appreciated with the benefit of the present teaching that the stimulation circuit 40 could comprise any other suitable signal generator 402. In particular, the amplifier and/or the filter 404 could comprise different components from those shown in FIG. 8.

Returning to FIG. 7, the controller 400 is operable to supply a signal (which is referred to herein as the “input signal” 405) to the signal generator 402. If the amplifier 403 of the signal generator 402 comprises a class-D amplifier, as shown in FIG. 8, the input signal 405 can be a pulse-width modulated binary signal. Alternatively, if the amplifier 403 of the signal generator 402 is not suited to receiving a pulse-width modulated signal, the input signal 405 can be an analogue signal, which can be generated by providing a digital value from the controller 400 to a digital-to-analogue converter (not shown in FIG. 7). The amplifier 403 is operable to amplify the input signal 405, in order to generate a signal 415 (which is referred to herein as the “amplified signal” 415). The power, voltage and/or current of the amplified signal 415 is preferably greater than that of the input signal 405 as a result of the amplification performed by the amplifier 403. The filter 404 is operable to attenuate one or more frequency components of the amplified signal 415. The output of the filter 404 (which is referred to herein as the “output signal” 416) is provided to the electrodes 50 via the terminals 412. As shown in FIG. 7, the signal generator 402 has two output terminals, and the output signal 416 is the voltage difference between the two output terminals of the signal generator 402.

The first means for measuring voltage 410 is connected to the terminals 412a and 412b. The first means for measuring voltage 410 is thereby operable to measure the voltage (i.e. the potential difference) between electrode 50a and electrode 50b.

The means for measuring current 406 comprises a resistor 407 and a second means for measuring voltage 408. The resistance of the resistor 407 is accurately known. The resistor 407 is connected in series between the output of the signal generator 402 and a first terminal 412a. Hence, the resistor 407 is in series with the electrodes 50. In use, the resistor 407 is in series with a human or animal body to which the electrodes 50 are connected. The second means for measuring voltage 408 is operable to measure the voltage across the resistor 407. When the signal generator 402 generates an output signal 416, an electrical current 422, 424 flows through the resistor 407, electrodes 50 and the body. The current through the resistor 407 is defined by Ohm's law:

I=V/R (2)

where I is the current (measured in amperes), V is the voltage across the resistor 407 (measured in volts) and R is the known resistance of the resistor 407 (measured in ohms). Thus, the means for measuring current is operable to measure the current through the resistor 407 using a measurement of the voltage across the resistor 407 and the known resistance. Since the resistor 407 is connected in series with the electrodes 50 and the body, the current through the resistor 407 is equal to the current through the body.

The first and second means for measuring voltage 408, 410 each preferably comprise a respective analogue-to-digital converter (ADC). Each analogue-to-digital converter is operable to convert an analogue input voltage to a digital value suitable to be input to the controller 400. Thus, the first means for measuring voltage 410 is operable to provide a first digital value to the controller 400, the first digital value being representative of the voltage between electrode 50a and electrode 50b. The second means for measuring voltage 418 is operable to provide a second digital value to the controller 400, the second digital value being representative of the current through the electrodes 50a, 50b. By using two analogue-to-digital converters, current and voltage can be measured simultaneously. The first and second means for measuring voltage 410, 408 are connected to the controller 400 by a respective bus 418, 420. Alternatively, each analogue-to-digital converter and the controller 400 can be provided in a single integrated circuit.

Preferably the controller 400 comprises a suitably programmed microprocessor or microcontroller. Alternatively, the controller 400 could be implemented using programmable logic, discrete logic gates or even a suitable analogue circuit. The controller 400 is operable to calculate the impedance of the body between the electrodes 50 when the stimulation pad 30 is in use.

The operation of the stimulation circuit 40 to measure impedance will now be described. In the following, it is assumed that the electrodes 50a, 50b of the stimulation pad 30 are electrically connected to a body. An input signal 405 is supplied to the signal generator 402 by the controller 400. In response to the input signal 405, the signal generator 402 generates an output signal 416. The output signal 416 causes a current (which is referred to herein as a “measurement current” 422) to flow. The measurement current 422 starts at the signal generator 402, flows through the resistor 407, then through the electrode 50a, then through the body, then through the electrode 50b and finally returns to the signal generator 402.

The current through the body is measured by the means for measuring current 406. More specifically, the current through the body is measured by measuring the voltage across the resistor 407 with the second means for measuring voltage 408, and dividing that voltage by the known resistance of the resistor 407 to provide a current measurement in accordance with equation (2). The voltage of the body between the electrodes 50 is measured by the first means for measuring voltage 410. The controller 400 calculates the impedance of the body in the region of the electrodes 50 using the measured current and voltage.

The equations used by the controller 400 to calculate impedance will now be described. The measurement current 422 can be either an alternating current or a direct current signal. If alternating current is used, the impedance is defined as:

Z=|V/I*e^{j(ø}^{_{V}}^{-ø}^{_{I}}⁾ (3)

where Z is the impedance of the body between the electrodes 50a, 50b (measured in ohms), V is the voltage across the electrodes 50a, 50b (measured in volts), I is the current through the body (measured in amperes), ø_{I} is the phase of the current, ø_{V} is the phase of the voltage, j is an imaginary number, and |x| denotes the amplitude of a variable x. Hence, the controller 400 can calculate the impedance of the body using a measurement of the current through the body, a measurement of the voltage of the body between the electrodes 50, a measurement of the phase difference between the current and voltage, and the relationship defined in equation (3).

If direct current is used, ø_{I} and ø_{V} are equal to zero and hence the impedance is equivalent to the resistance defined by Ohm's law:

R=V/I (4)

where R is the resistance of the human or animal body between the electrodes 50a, 50b (measured in ohms), V is the voltage across the electrodes 50a, 50b (measured in volts) and I is the current through the body (measured in amperes). Hence, the controller 400 can calculate the impedance of the body using a measurement of the current through the body, a measurement of the voltage of the body between the electrodes 50, and the relationship defined in equation (4).

The impedance measurement represents the impedance of the region of the body that is local to the electrodes 50. Hence, the impedance measurement provides information on the conditions in the region local to the electrodes, but not on the body as a whole. If necessary, the overall impedance of the body can be estimated by combining local impedance measurements taken at several body locations.

The operation of the stimulation circuit 40 to apply electrical stimulation will now be described. In the following, it is assumed that the electrodes 50a, 50b of the stimulation pad 30 are electrically connected to a body. An input signal 405 is supplied to the signal generator 402 by the controller 400. In response to the input signal 405, the signal generator 402 generates an output signal 416. The output signal 416 causes a current (which is referred to herein as a “stimulation current” 424) to flow. The stimulation current 424 starts at the signal generator 402, flows through the resistor 407, then through the electrode 50a, then through the body, then through the electrode 50b and finally returns to the signal generator 402.

When applying electrical stimulation to the body, the controller 400 is preferably operable to control the output signal 416 based upon the measured impedance of the body. For example, by controlling the amplitude and/or the duration of the input signal 405, the controller 400 can control the amplitude and/or duration of the output signal 416. The controller 400 can also stop supplying an input signal 405 to the signal generator 402, so as to stop the output signal 416 being generated and thereby stop electrical stimulation being applied to the body.

The measured impedance may vary due to perspiration and/or improper placement of the pad 30. The presence of perspiration can create a low-impedance conducting path across the surface of the user's skin, which will cause the measured impedance to decrease. Improper placement of the pad 30 can result in poor electrical contact between the electrodes 50 of the pad 30 and the body, which will cause the measured impedance to increase. Physiological effects, such as an increase in the blood volume of muscles, may also cause small changes in the measured impedance. The controller 400 can adjust the amplitude of the electrical stimulation to compensate for changes in the measured impedance. For example, the controller 400 can increase or decrease the amplitude of the voltage of the output signal 416 (or the amplitude of the stimulation current 424) to ensure that the level of electrical stimulation that is actually delivered to the body remains constant.

The main reason for measuring the impedance of the body is to ensure the safety of the user during electrical stimulation. For example, if perspiration were to cause the impedance between the electrodes 50 of the pad to decrease whilst the voltage of the output signal 416 remained constant, the stimulation current 424 applied to the body would increase. To prevent the stimulation current 424 increasing to a level that could be harmful to the user, the controller 400 can stop the output signal 416 being generated if the impedance of the body decreases below a first threshold value. As another example, to avoid electrical stimulation being applied in the event of poor electrical contact between the electrodes 50 and the body, the controller 400 can stop the output signal 416 being generated if the impedance of the body increases above a second threshold value. The controller 400 can also generate an alert (such as an audible alert and/or a visible alert) if the impedance decreases below the first threshold value or increases above the second threshold value.

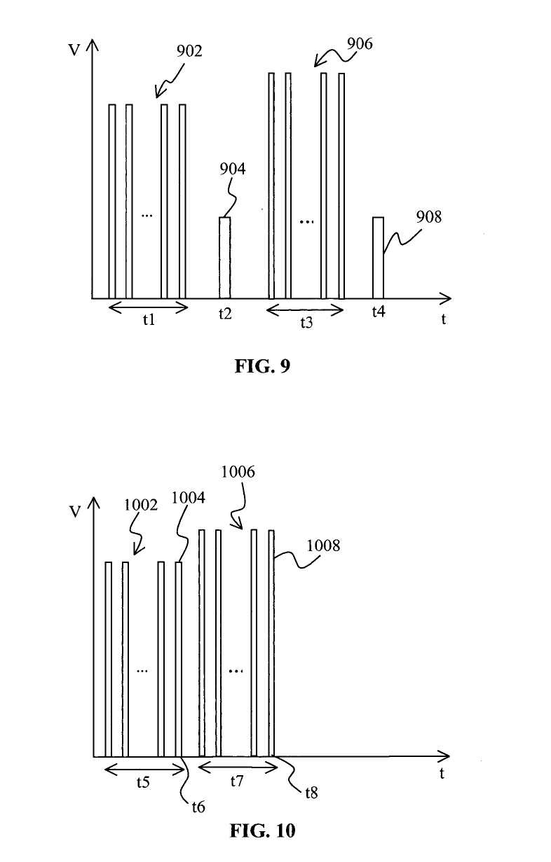

FIG. 9 illustrates the use of the stimulation circuit to apply electrical stimulation and to measure the impedance of the body. FIG. 9 is a graph (not drawn to scale) showing the voltage applied to the body on the vertical axis, and time on the horizontal axis. A first series of electrical stimulation pulses 902 is applied to the body during time interval t1. A measurement voltage signal 904 is applied at time t2, and the impedance of the body is measured whilst the measurement voltage signal 904 is applied. The amplitude of the measurement voltage signal 904 is smaller than that of each pulse in the first series of electrical stimulation pulses 902. A second series of electrical stimulation pulses 906 is applied to the body during time interval t3. The second series of electrical stimulation pulses 906 is preferably controlled based upon the measured impedance. As shown in FIG. 9, the amplitude of each pulse in the second series of electrical stimulation pulses 906 is greater than that of each pulse in the first series of electrical stimulation pulses 902. A further measurement voltage signal 908 is applied at time t4, and the impedance of the body is measured whilst the further measurement voltage signal 908 is applied.

When the impedance of the body is being measured, the amplitude of the output signal 416 is chosen so as to be too small to cause nerve stimulation. This prevents muscle contraction whilst impedance is being measured, and thereby improves the accuracy of the impedance measurement. A suitable amplitude for the output signal 416 can be empirically determined. Thus, as shown in FIG. 9, the amplitude of each measurement voltage signal 904, 908 is smaller than the amplitude of the electrical stimulation pulses 902, 906.

FIG. 9 illustrates that electrical stimulation is applied at a different time from that at which the impedance of the body is measured. However, the stimulation circuit 40 described herein also allows impedance to be measured at the same time as electrical stimulation is applied. In order to apply electrical stimulation and measure impedance simultaneously, the means for measuring current 406 measures the current through the body whilst the stimulation current 424 is being applied to the body, and the first means for measuring voltage 410 measures the voltage of the body between the electrodes 50 whilst the stimulation current 424 is being applied to the body; there is no need for a separate measurement current 422.

FIG. 10 illustrates the use of the stimulation circuit to apply electrical stimulation and measuring impedance simultaneously. FIG. 10 is a graph (not drawn to scale) showing the voltage applied to the body on the vertical axis, and time on the horizontal axis. A first series of electrical stimulation pulses 1002 is applied to the body during time interval t5. The impedance of the body is measured at time t6, during the final pulse 1004 of the first series of pulses 1002. A second series of electrical stimulation pulses 1006 is applied to the body during time interval t7. The second series of electrical stimulation pulses 1006 is preferably controlled based upon the measured impedance. As shown in FIG. 10, the amplitude of each pulse in the second series of electrical stimulation pulses 1006 is greater than that of each pulse in the first series of electrical stimulation pulses 1002. The impedance of the body is measured again at time t8, during the final pulse 1008 of the second series of pulses 1006. By measuring impedance at the same time as electrical stimulation is applied, the need for separate measurement voltage signals (as denoted by reference signs 904 and 908 in FIG. 9) is eliminated, which advantageously avoids electrical stimulation being interrupted to measure impedance.

As described above, electrical stimulation is applied to a body using the same electrodes 50 that are used to measure impedance of the body. The shared use of a single pair of electrodes 50 is advantageous because it allows impedance to be measured at the region of the body to which electrical stimulation is applied. In particular, this allows the detection of undesirable low-impedance conducting paths caused by perspiration, and allows action to be taken to prevent those conducting paths causing unsafe levels of electrical stimulation being applied to the body. The shared use of a single pair of electrodes also allows the quality of electrical contact between the electrodes and the body to be determined, and allows action to be taken if the quality of electrical contact is poor. The shared use of a single pair of electrodes also allows the electrical stimulation to be controlled based upon the local impedance at precisely the region of the body to which stimulation is applied. Additionally, the shared used of a single pair of electrodes 50 eliminates the need for separate sets of electrodes to measure impedance and to apply electrical stimulation. This simplifies the stimulation circuit 40 and results in a stimulation pad 30 that is compact and simple to manufacture. Furthermore, the shared use of a single pair of electrodes makes the stimulation system easier to use, by avoiding the need to apply separate sets of electrodes to the body in order to perform electrical stimulation and impedance measurement.

If the pad 30 comprises a heating element, the controller can preferably control the temperature of the heating element based upon the measured impedance of the body. This also has the advantage of allowing the therapeutic treatment to be adapted based upon how the user's body is responding to the therapy. The temperature of the heating element can be increased, decreased or maintained at its current level based upon the measured impedance.

Whilst the stimulation circuit 40 is described above as being a component of console 20, it is also possible to include some or all of the functionality of the stimulation circuit 40 in the stimulation pad 30. For example, the stimulation pad 30 could comprise the means for measuring current 406, the first means for measuring voltage 410 and a means for calculating impedance. In this example, the stimulation pad 30 could communicate a digital or analogue value representative of the impedance of the body to the console 20 via the cable 60. The present invention preferably encompasses arrangements in which some or all of the functionality of the stimulation circuit 40 is implemented in the stimulation pad 30.

It will be understood that the invention has been described above purely by way of example, and that modifications of detail can be made within the scope of the invention.

1. An apparatus comprising:
means for applying electrical stimulation to a human or animal body via a pair of electrodes; and
means for measuring impedance of the body between the pair of electrodes.

2. An apparatus in accordance with claim 1, further comprising means for controlling the electrical stimulation based upon the impedance measured by the means for measuring impedance.

3. An apparatus in accordance with claim 2, wherein the means for controlling the electrical stimulation is operable to adjust the amplitude of the electrical stimulation applied to the body.

4. An apparatus in accordance with claim 3, wherein the means for controlling the electrical stimulation is operable to adjust the amplitude of the electrical stimulation applied to the body to compensate for variations in the impedance measured by the means for measuring impedance.

5. An apparatus in accordance with any of claims 2 to 4, wherein the means for controlling the electrical stimulation is operable to stop electrical stimulation being applied to the body if the impedance measured by the means for measuring impedance is less than a first threshold impedance value.

6. An apparatus in accordance with any of claims 2 to 5, wherein the means for controlling the electrical stimulation is operable to stop electrical stimulation being applied to the body if the impedance measured by the means for measuring impedance is greater than a second threshold impedance value.

7. An apparatus in accordance with any of the preceding claims, the apparatus further comprising a pad for placement on the body, wherein the pad comprises the pair of electrodes.

8. An apparatus in accordance with any of the preceding claims, wherein the means for measuring impedance comprises a first means for measuring voltage, the first means for measuring voltage being operable to measure the voltage between the pair of electrodes.

9. An apparatus in accordance with any of the preceding claims, wherein the means for measuring impedance comprises a means for measuring current, the means for measuring current being operable to measure the current through the electrodes.

10. An apparatus in accordance with claim 9, wherein the means for measuring current comprises:
a resistor arranged to be connected in series with the electrodes; and
a second means for measuring voltage, the second means for measuring voltage being operable to measure the voltage across the resistor.

11. An apparatus in accordance with claim 10 as dependent upon claim 8, wherein the means for measuring impedance comprises means for calculating impedance using the voltages measured by the first and second means for measuring voltage.

12. An apparatus in accordance with any of claims 9 to 11, wherein the means for measuring current is operable to measure current through the electrodes whilst the means for applying electrical stimulation is applying electrical stimulation to the body.

13. An apparatus in accordance with any of claims 1 to 11, wherein the means for measuring impedance comprises means for applying a measurement signal to the body, the means for measuring impedance being operable to measure impedance of the body whilst the measurement signal is being applied.

14. An apparatus in accordance with claim 13, wherein the amplitude of the measurement signal is chosen to prevent muscle contraction.
